(19) United States
(12) Patent Application Publication
(10) Pub. No.: US 2021/0295728 A1
(43) Pub. Date: Sep. 23, 2021

(54) Artificial Intelligent (AI) Apparatus and System to Educate Children in Remote and Homeschool Setting

(71) Applicants: Elnaz Sarrafzadeh, San Jose, CA (US); Rong Cheng, Shenzhen (CN)

(72) Inventors: Elnaz Sarrafzadeh, San Jose, CA (US); Rong Cheng, Shenzhen (CN)

(21) Appl. No.: 16/922,451

(22) Filed: Jul. 7, 2020

Related U.S. Application Data
(60) Provisional application No. 62/992,044, filed on Mar. 19, 2020.

(51) Int. Cl. G09B 7/08; G09B 5/06; G06K 9/62; G10L 15/08; G06N 20/00

(52) U.S. Cl. CPC G09B 7/08; G09B 5/06; G06N 20/00; G10L 15/083; G06K 9/6256

(57) ABSTRACT

An apparatus for educating children comprising inserting a template into a cover for a protective helmet so that the cover is stretched and once the template is inserted into the cover it forms a disk-shaped assembly; providing at least one light tubes wherein the light tubes are to be affixed to said cover; determining the arrangement of said light tube to be affixed on to said cover; determining a plurality of tabs wherein said tabs will be sufficient to hold said light tube; opening at least two opposing holes on said cover around said tabs whereby forming said tabs on said cover; placing the disk-shaped assembly on top of a bottom plate of a heat press; placing a patch on top of the disk-shaped assembly; placing a heat press cover sheet on top of the disk-shaped assembly; pulling down an upper plate of the heat press so that the heat press cover sheet makes contact with the upper plate of the heat press when the heat press is closed; and pulling up the upper plate.

### INCORPORATION BY REFERENCE

This application claims the benefit of priority under 35 U.S.C. 119(e) to the filing date of U.S. Provisional application No. 62/992,044, entitled “Apparatus, Robot and System to Educate Children on Health and Safety,” which was filed on Mar. 19, 2020, and which are incorporated herein by reference in their entirety.

### FIELD OF INVENTION

The present invention relates to children's educational devices. Specifically, the present invention is directed to an artificial intelligence (“AI”) enabled device that functions as a both child's companion but also as a self-operating instructor that presents and teaches the child different basic developmental subjects and skills from either a library of learning modules which are programed locally but the device also contemplates use in the context of distant learning.

### BACKGROUND OF THE INVENTION

It has long been appreciated that child development is a complex, multi-faceted, and fascinating process, and every new parent labors to balance raising a healthy child in an increasingly hectic and now uncertain world, struggling to balance their own lives, time, and resources as best they can. Now particularly, many households require that both parents work, which is somewhat a frustration in itself as the daycare bills can often quickly eat though one person's paycheck such that the reward of the second income is significantly diminished. Separate from the economic cost alone, many parents have fears that the care their child receives in their absence may not be the best, and unfortunately these fears are somewhat rational as the news reminds all of us some of the horrors of the world we all wished weren't true when the story of the now turns to another story of daycare malpractice. Unfortunately, other families are single parent households where the lack of resources is often only amplified. Some of these impacted households can depend on family, but this is not always to the benefit of the child or the grandparents or other extended family.

As such, in an increasingly busier world that now has the principles of robotics, and artificial intelligence understood that an automated companion be considered, such that a parent may be relieved the endless daycare tuition costs, as well as the other uncertainties that naturally come with leaving an unattended child under another person's care. Separate from alleviating a parent's general childcare anxieties a key component of childcare is a child's basic skills and educational development.

It has been known that young children have a very short attention span, albeit the select song or book that they will listen to again and again ad nauseam. Thus, educating young children requires some well-thought, well-designed, structured programs to engage their attention as long as short attention span may be, and continually train that attention span to be slightly longer, and to nudge them in a right direction towards other development progress milestones. To date, much of the educational materials marketed and readily available to parents, who are the de facto primary educators of young children, has been through printed books, the internet and its digital offerings, and audio-visual material such as children's television programming. While some of this material is quite good and informative, given the way that technology has developed, it is often rather passive and is rarely interactive. Typically, the child is being spoken to or presented images, and there is little if any feedback to register a child's comprehension.

There are many aspects to the development of young children, such as physical and physiological development, emotional development, and psychological development. It has been found that interactive educational materials can be designed and tailored to aid in teaching and improving the development of all of these different metrics. It is also known that learning is often more effective when it is interactive, personalized and that people have a better retention when the subject matter is made interesting and memorable versus mere presentation of the materials. It is also well appreciated that interactive and guided instruction can account for hurdles that a child might experience in grasping particularly difficult concepts whereas the presentation method of teaching often leaves some children behind and frustrated, which only complicates as lessons progress without addressing these gaps.

Meanwhile, it has been found that manufacturers of products for young children through trial and error and the guidance of product development have valuable insight now into how to communicate information to young children in a manner that is informative, fun, and easy to remember. As mentioned, interactive and fun communication offers several advantages over passive and non-interactive information presentation to the child, but it has also been found that parents of young children themselves appreciate the interactive information as well and will naturally participate in and aid the teaching of their young children because the lessons are fun and, thus, very effective as memory aids for the parents themselves. In observing the child's lesson, a parent themselves are passively learning how to teach their child long after the lesson has ended. While, the interactive lessons having sounds and visual cues better develop young children's senses and brain, they also are catchy and fun, and inevitably some of these lessons are incorporated into the day to day parent-child interaction which only reinforces the subject matter that much more effectively

As can be implied, interactive educational devices have an enormous advantage where parents have limited time to accompany or teach their children, or they do not have the skills and knowledge themselves necessary to teach. In these cases, in the past, affluent parents resorted to childcare and/or tutoring centers, or private tutors, which can reasonably be expected to be more and more expensive as the notoriety of a particular school rises, being of such significance that often a particular school may dictate where the parents themselves choose to purchase a home. While tutoring somewhat frees a parent from perhaps living in a particular place, tutoring services are understandably limited in their own ways; most tutoring services are not for all ages, most only accept first grade or older students. Because of travel and time constraints, most tutoring centers limit themselves to teaching core subjects which can be broken into modules that fit the business needs of the tutoring center, focusing on basic math, science, or reading. Other developmental skills, such as physical and physiological, emotional, or psychological development, are not traditionally tutored in this format. As such, and as can be heard from anecdotal stories of children who were home schools, this insufficiency is sometimes detrimental to the holistic development and growth of young children, with many of these homeschooled taught excelling in testable subject matter but still having gaps in these other neglected subjects. Consequently, there is now a large demand for modern solutions for young children's education and training of critical early childhood skills, such as health and safety conduct, early language development, and so on. As can be expected, an entire market's supply of children's educational self-help, interactive devices or tools has emerged in response to these problems over the years. Unfortunately, once the veneer of marketing is peeled away, while maybe these teaching tools advertise themselves as interactive really aren't, or are severely limited as to the freedom of interaction that is allowed or even possible. Instead of one-directional (from the device to children) content delivery, interactive devices if they are truly to be interactive ought to allow a child's input and participation in the lesson, and provides real-time feedback to re-enforce or adjust the lessons as appropriate to that specific child. That said, when examining nearly all of these devices, most of the devices, while providing electronically produced sounds or images, are still one-directional when taken as a whole. That is, the content is given to the children, and while there may be these bells and whistles going off giving a parent the illusion of interactivity, the lessons still progress in a linear fashion and are the same from child to child. As can be expected, because the device is not actively engaging the children, the materials and devices cannot hold their attention for long once the child habituates to the formerly novel sounds and images and many of these devices lose their efficacy and are set aside by the child to the frustration of the parents

Therefore, parents are effectively buying advice which was advertised as educational but quickly becomes just another toy for the child.

Another problem that overlaps some with issues of attention span with other novel issues wholly by itself is that ultimately children are limited by the modules or libraries available and may only learn whatever content is installed in the device. A child who is not interested in a particular subject coupled with a teacher that cannot respond to those cues and change tactics is easily defeated by the youngest child. On the other end of the spectrum, a child who is blessed with a voracious appetite for learning may quickly go through these canned teaching modules and become bored because they are not being challenged. This ultimately means that once the child is tired of the device (toy), the device will end up in storage. This method of delivering educational content benefits the manufacturers because they know there will be a new round of exhausted, confused, new parents to sell more units of the devices. Other less observant parents will simply buy a new device wasting money to achieve predictable results. Some children may even like the device and develop an emotional attachment to it, so it does not help to replace the device, yet the child is not being actively challenged or taught anything through the device.

Apart from the deficiencies in the learning goal of the current state of education devices, some of these devices miss the mark in entirely other metrics. In one aspect, some of current educational devices or systems are unintuitive and hard to use, and/or require some computer system knowledge to operate. As such, what was originally meant to be a time saving device actually requires the attention of the parent to use. When the child needs their parents' help to launch and/or operate the devices, this does not free up parents to do other chores or work as they were originally expecting for themselves, so to that end, the devices fail to both teach and to save the time of the parent.

In light of these drawbacks, it is then desirable to have an educational device that has both an appealing appearance to young children, but also hosts a powerful educational content library to offer a child, a powerful remote delivery system of content such that the device is relatively self-operating, and critically a smart interactive system to retain a young child's attention and to keep the child engaged in positive and productive activities.

Such a device would carry additional benefits as a device that could more robustly respond to a child's needs would enable the AI device to also function as a more reliable distant learning tool for a young child. The current education model requires young children to go to school for learning, often away from their homes, requiring frequent commuting. Along with this a parent is expected to organize time for their child's participation in daytime, and sometimes evening or week-end classes, programs, sports activities and the like. While these social and extra-curricular activities have their own benefits, as the adage goes, time is a fixed resource. As such, when these other activities ramp up in frequency or commitment costs, this can significantly increase the cost of study for some and limit the time available to study for others.

It is also recognized that many segments of the population, especially those with aspirations of college and scholarships are interested in improving their qualifications to stand out in a crowded field and hopefully improve their chances of acceptance into the schools of their choosing and potentially receiving financial awards. Unfortunately, those that need these scholarships are traditionally those which are already disadnessvantaged in other ways: financially strained households, working households where the child may be raising themselves or helping with raising other children, or children which are physically handicapped or other genetic disadnessvantages.

The contemplated device as described would be an enormous resource in that it would facilitate a greater quality of remote and self-study courses such that those either have disadnessvantages for time or money or both might be able to more reasonably look to and depend on remote courses to fill their educational needs. In tracing the requirements for distance education using technology, a number of systems and methods for teaching, utilizing electronic means of data processing, transfer and communication, have been developed. Currently, remote courses rely on and validate themselves nearly entirely through the tracking of various metrics, mostly attendance and performance on assignments and tests. Without overexplaining the obvious, these programs are particularly susceptible to fraud and cheating. Further, much of the instruction mirrors the earlier discussed problem in that much of the teaching is one way, or a limited question format. Further, because of the limited time allotted, many of these questions may be overlooked or not really given the attention that a 1:1 tutor type instructor would be able to provide. For this purpose, it is necessary, and now commercially viable, to develop a distance learning education system for young children, including the whole program of the studies, which should be the same as in the case of traditional studies and should ensure a high level of education, with frequent contacts between the students and the lecturers. Thanks to this, it will be possible for the students to gain the same knowledge and qualifications as in traditional studies.

Finally, as the world is still recovering from the recent pandemic, it has been considered the value that could be gained from using the AI device as a minimally invasive self-care and well-being monitoring tool for a young child.

There are lots of reasons causing illness and regrettably the sudden death of a children. Although the reasons are complicated, sudden death is often either directly caused or at the very least exacerbated by negligence which has occurred in the taking care of a children. Significantly less tragic but still of concern are the more minor illnesses like colds that a small child can be expected to encounter but are made more severe by easily preventable conditions.

Many parents would be surprised to know that some of the most basic things can adversely impact their child's self-care and well-being. For example, it is easy for an adult to forget that babies and small children simply lack the body fat percentage that an older child or adult has. This thin layer of fat helps insulate adults against slight differences in the temperature in an environment. As such, the temperature that a thermostat is set to is going to affect a children or small child more than it would the parent, and whereas an adult will sleep through a cold night and perhaps wake up feeling cold, the children will have a rougher night, and in instances where the children is sick, this discomfort will exacerbate conditions. Whereas an adult cannot realistically be expected to watch over a child through the night to notice discomfort, the contemplated AI device can observe conditions, and before the parent is notified, the AI device itself can first prompt the children to self-help, whether that be seeking the quilt because the AI device can pick up cues from the children that he or she is cold. Similarly, the AI device could coax the child to kick themselves out from under a quilt if they are identified to be hot.

Further, and as hinted at previously, because the AI device is monitoring the child's condition, it can also notify the parent sooner for some more urgent conditions or symptoms. For example, an initial period of fever is rarely discovered when the children is still energetic enough and is also wearing too much that he or she does not sweat easily. An AI device may take some of these excuses into consideration but would be expected to also be much more clinical and unbiased in detecting abnormal conditions or symptoms. Whereas some parents may be more optimistic, or simply too tired themselves to notice, the AI device will be able to consistently be monitoring the child's self-care and well-being.

This same technology could similarly provide relief for the other age range where self-care may be difficult, the elderly, the handicapped, or those that may be recovering from a traumatic injury. Many of the same situations would apply, however instead of the parent receiving help, it may be the elderly persons children, or perhaps the nurses that have been charged with the care of a large number of patients and would need similar assistance. For the existing drawbacks, it is desirable to have a monitor device that has an appealing appearance to young children.

### Objective of the Invention

The main purpose of the utility model is to solve the expandability and ease of use of children's educational robots, increase children's interest and interaction with the product, and meanwhile, the invention also has the characteristics of easy implementation and low cost.

It is also an objective for the invention to develop an AI companion Robot to young children for education purposes. It is also an objective for the invention to develop AI companion Robot to young children for distance and remote education purposes. It is also an objective for the invention to develop AI companion Robot to young children for self-care and well-being monitoring of young children.

### BRIEF DESCRIPTION OF THE DRAWINGS

These and other features and advantages of the invention will not be described with reference to the drawings of certain preferred embodiments, which are intended to illustrate and not to limit the invention, and in which:

FIG. 1 illustrates an embodiment of the current invention.

FIG. 2 illustrates another embodiment of the current invention comprising a charging pad.

FIG. 3 illustrates a charging pad of another embodiment of the current invention.

FIG. 4 illustrates another charging pad of yet another embodiment of the current invention.

FIG. 5 illustrates another embodiment of the current invention, comprising a power socket and method of delivering power to the system.

FIG. 6 illustrates a flowchart of an exemplary system of the current invention.

FIG. 7 illustrates a flowchart of an exemplary system of the current invention.

FIG. 8 illustrates a flowchart of an exemplary system of the current invention.

FIG. 9 illustrates a flowchart of an exemplary personality trait learning and smart educational content based on personality traits.

FIG. 10 illustrates an embodiment of the current invention.

FIG. 11 illustrates an embodiment of the current invention.

### SUMMARY OF THE INVENTION

In one aspect of the invention, an apparatus for educating children is disclosed comprising: an anime-like body comprises an upper and lower body and two feet upon which the anime-like body stands; wherein the upper body is outfitted with an interchangeable cap that comprises two decorative ears; wherein the upper body further comprises a prominent camera that is connected and controlled by a central processing system disposed inside the anime-like body; wherein the camera further comprises a facial recognition system that triggers the camera to capture a motion video; wherein the central processing system processes the motion video; wherein the lower body further comprises a LCD for displaying educational content, warning or feedback messages, that is controlled by the central processing system; wherein the apparatus further comprises a charging pad upon that the anime-like body stands while it is being charged; wherein the charging pad further comprises a home button, a right input button, and left input button; wherein the home, left and right button is made of clear and soft material that covers an LED light array underneath; wherein the LED light array is controlled by the central processing system to display different colors as visual cues; wherein the charging pad further comprises a shaped depression that one-way fits the two feet; wherein the shaped depression further comprises two sets of electrical pins disposed where the two feet to be set; wherein the two feet further comprises charging plates at the two feet's bottoms; wherein the two sets of electrical pins engage the charging plates; wherein the two feet further comprises two two-way audio unit to pick up and deliver sound content; wherein the LCD displays a question as the apparatus requests a respond through the two-way audio unit; wherein pressing one of the home, left, or right button answers the question; wherein the apparatus further comprises a first two-way wireless communication counterpart; wherein the charging pad further comprises a second two-way wireless communication counterpart; wherein the charging pad further comprises a power socket that receives a DC jack; wherein the apparatus further comprises an inertial measurement unit (IMU) disposed inside the apparatus.

In one embodiment, the sounds received by the two-way audio unit are processed by an integrated circuit (IC) that is a system on chip (SOC) ICB. In one embodiment, the interchangeable cap is a protective cover to protect the upper body. In one embodiment, the interchangeable cap has an appealing design, shape, and color to attract and retain young children's attention. In one embodiment, the apparatus further comprises an electromagnetic inductive charging component. In one embodiment, the communication signals forth and back between the apparatus and the charging pad are transmitted via a wireless component. In one embodiment, the charging pad further comprises at least one magnet that holds the two feet in place and keeps the apparatus standing while it's being charged. In one embodiment, the IMU is comprised of a gyroscope, an accelerometer and a magnetometer.

In another aspect of the invention, a children education apparatus is disclosed comprising: a capsule shape housing unit having a detachable cover part and a feet part wherein the capsule shape housing unit is comprised of an upper capsule part and a lower capsule part wherein the upper capsule part having a forward facing surface and a rearward facing surface wherein the upper capsule part is further comprised of a hemispherical dome part wherein the hemispherical dome part is positioned in vertical fashion along the forward facing surface; the hemispherical dome part houses a digital camera and the detachable cover part is comprised of flexible material and wraps around the upper capsule part in its entirety except wherein the detachable cover part has an opening allowing the hemispherical dome part to protrude through the detachable cover part and the detachable cover part further comprising a vertical standing part for decorative purpose; the lower capsule part is connected of the feet part wherein the feet part is comprised of a first foot and a second foot and the first foot and second foot are of equal length, wherein the lower capsule part further comprises a digital display wherein the digital display is rectangular in shape and wherein the digital display is a LCD, wherein the lower capsule part further comprises at least one microphone, at least a speaker, wherein the capsule shape housing unit further comprises a inertia momentum unit, at least one battery, a computing processing unit. In one embodiment, the children education apparatus unit further comprise a base apparatus wherein the base apparatus comprises a battery charging unit and a recess to receive the first foot and the second feet and charges the battery when the first foot and the second foot rests on the recess; the base apparatus further comprising a plurality of input buttons wherein the input buttons provides input controls the children education apparatus. In one embodiment, the input buttons provides input controls to the children education apparatus when the first foot and the second foot rests on the base apparatus. In one embodiment, the input buttons provides input controls to the children education apparatus when the first foot and the second foot are not resting on the base apparatus. In one embodiment, the vertical standing part is comprised of 2 triangular shape standing parts. In one embodiment, the vertical part is comprised of 2 vertical standing reverse teardrop shape parts. In one embodiment, the vertical part is comprised of a triangular shape standing part having a circular ball shape part attached to the at the tip of the triangular shape standing part. In one embodiment, the capsule housing unit further comprises a temperature sensor.

In yet another aspect of the invention, a children education apparatus is disclosed comprising a central processing unit, an inertia momentum unit, a battery, a camera, a LCD unit, and an education platform power by the central processing unit wherein the education platform is comprised of plurality of children education content and delivers the plurality of children education content to the LCD unit where the education platform detects a movement of the children education apparatus via the inertia momentum unit and determines which one of the plurality of children education content to the LCD.

In another aspect of the invention, a children education apparatus is disclosed comprising a central processing unit, an inertia momentum unit, a battery, a camera, a microphone, a temperature sensor, a LCD unit, and an education platform power by the central processing unit wherein the education platform is comprised of plurality of children education content and delivers the plurality of children education content to the LCD unit where the education platform is comprised of a video data analysis module, a voice data analysis module, a movement data analysis module and a temperature data analysis module wherein the camera records one or more video data and the microphone records one or more records one or more audio data and the inertia momentum unit records one or more movement data and the temperature sensor records one or more temperature data. In one embodiment, the video data analysis module analyzes the video data and determines which one of the plurality of children education content to deliver to the LCD. In one embodiment, the audio data analysis module analyzes the audio data and determines which one of the plurality of children education content to deliver to the LCD. In one embodiment, the temperature data analysis module analyzes the temperature data and determines which one of the plurality of children education content to deliver to the LCD. In one embodiment, the movement data analysis module analyzes the movement data and determines which one of the plurality of children education content to deliver to the LCD. In one embodiment, the video data analysis module analyzes the video data and determines an emotion characterization of a user and determines which one of the plurality of children education content to deliver to the LCD base on the emotion characterization. In one embodiment, the audio data analysis module analyzes the audio data and determines an emotion characterization of a user and determines which one of the plurality of children education content to deliver to the LCD base on the emotion characterization.

### DETAILED DESCRIPTION OF THE DRAWINGS

The invention will be described in the context of a preferred embodiment. The following detailed description and the appended drawings describe and illustrate exemplary embodiments of the invention solely for the purpose of enabling one of ordinary skill in the relevant art to make and use the invention. As such, the detailed description and illustration of these embodiments are purely exemplary in nature and are in no way intended to limit the scope of the invention, or its protection, in any manner. It should also be understood that the drawings are not to scale and in certain instances details have been omitted, which are not necessary for an understanding of the present invention, such as conventional details of fabrication and assembly.

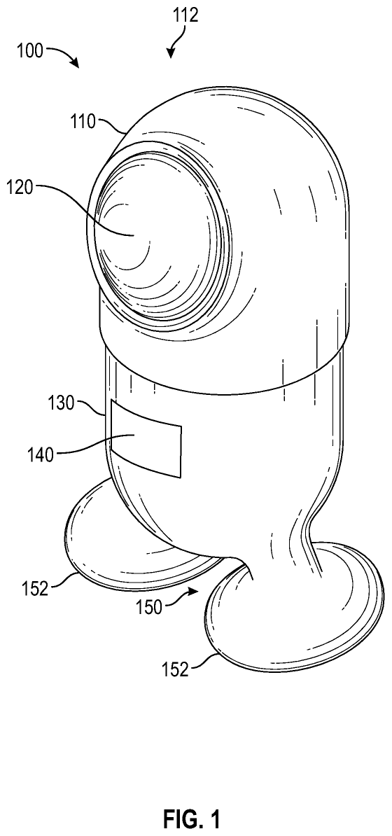

FIG. 1 illustrates an embodiment 100 of the current invention comprising a body that looks like an anime character which, in turn, comprises an interchangeable cap 110 that comprises two decorative ears 112. It is appreciated that the interchangeable cap 110 can be of different colors and designs and/or shapes. This interchangeable cap 110 is also contemplated to be protective cover to protect the top half of the embodiment 100. It also has an appealing design, shape, and color to attract and retain young children's attention. The embodiment 110 further comprises a prominent camera 120 disposed on the top half of the embodiment. The camera is connected and controlled by a central processing system from inside the embodiment's body. The camera 120 further comprises a facial recognition system to detect a child's head and hand motions, and triggers the camera to capture the child's motion video and the central processing system to process the video. Where the child touches her eyes, nose, or mouth, the embodiment 100 would speak through its speakers disposed a reminder of not touching one's eyes, nose, or mouth with one's dirty hands. The embodiment 100 further comprises a LCD 140 for educational video content that is disposed of on the lower half 130 of the embodiment. The LCD 140 is controlled by the central processing system to display educational content, warning or feedback messages. The embodiment 100 further comprises two feet 150 upon which the embodiment stands. As disclosed hereinabove, the two feet 150 further comprises two two-way microphones 152 to pick up and deliver sound content.

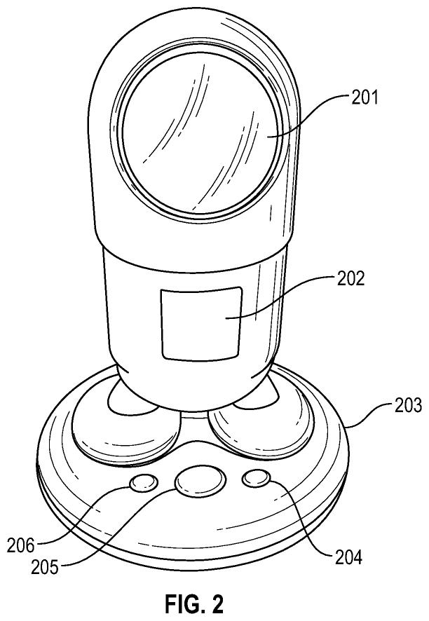

FIG. 2 illustrates an embodiment 200 of the current invention comprising a body that looks like an anime character which, in turn, comprises a camera 201 and a LCD 202. This embodiment 200 further comprises a charging pad 203 where the embodiment's body stands on while being charged. The charging pad further comprises a home button 205, a right input button 204, and left input button 206. The home, left and right buttons are made of clear and soft material to cover an LED light array underneath. The LED array can be controlled to display different colors as visual cue to a child. In operating mode, the embodiment 200 will display a picture on the LCD 202 with a question and prompt the user to respond but inputting either home button 205 or select either input buttons 204 or 206. In one embodiment, various LED colors will light up button 205, button 206, or button 204. The use of color LEDs to light up the buttons in connection to the images displayed on the LCD 202 as a way of communicating and educating young children. The young children interact with the embodiment 200, such as answering multiple-choice questions, identifying colors, directions, playing games, etc., by pressing the correct button. The embodiment can detect the children's input by checking the received unique button identification number. The communication data received is processed by an MCU, that is an integrated circuit (IC) that is, in this embodiment, a system on chip (SOC) ICB. The embodiment 200 further comprises a wireless communication component that comprises two two-way communication counterparts. One of these resides in the embodiment's body and the other in the charging pad.

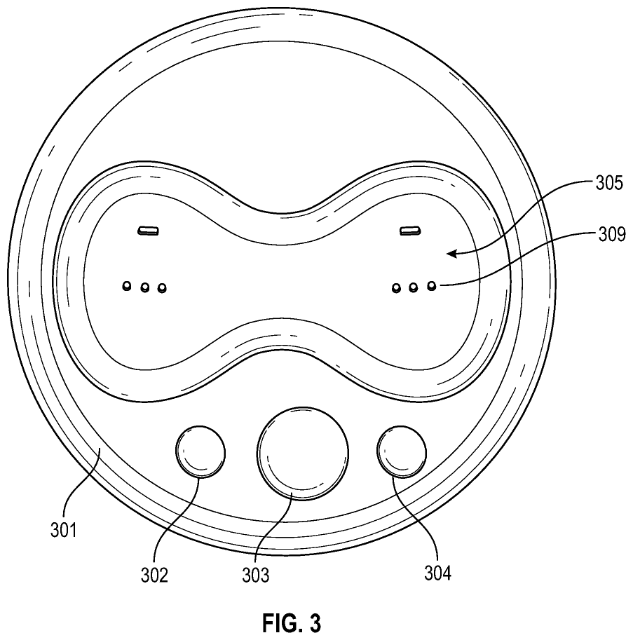

FIG. 3 illustrates an embodiment 300 of the current invention comprising a charging pad 301 that, in turn, comprises a home button 303, a left input button 302, and right input button 304. The charging pad 301 further comprises a shaped depression 305 in which the robot's feet are set in. It's contemplated that the shape of the depression 305 allows only one way of setting the robot's feet; the robot cannot stand balanced in the wrong way. The depression 305 further comprises two sets of electrical pins 306 disposed where the robot's feet to be set. These electrical pins will engage the charging plates at the bottom of the robot's feet. It is contemplated that another embodiment will comprise an electromagnetic or inductive charging component that wirelessly charges the robot's batteries. In this embodiment, since there is no electrical contact between the charging pad and the robot, the communication data forth and back between the two are sent and received via a wireless component.

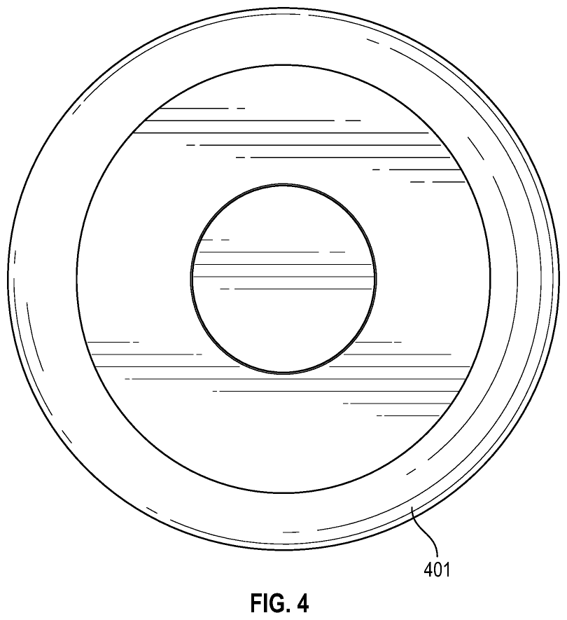

FIG. 4 illustrates another embodiment 400 of the current invention comprising a charging pad 401 comprises a electromagnetic or inductive charging component (not shown; enclosed inside the charging pad). The charging pad 401 further comprises at least one magnet that holds the robot's feet in place and keep the robot standing while it's being charged. The magnet force eliminates the need for a shaped depression as disclosed above.

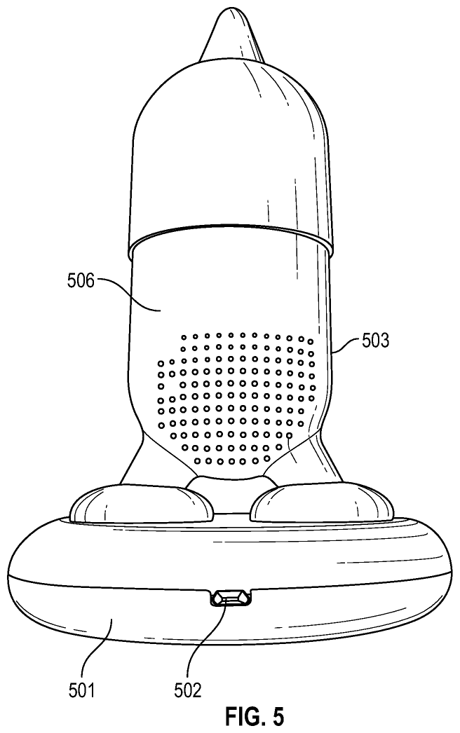

FIG. 5 illustrates another embodiment 500 of the current invention comprising a charging pad 501 that comprises a power socket 502 that in turn receives a DC jack. The embodiment further comprises an inertial measurement unit (IMU) embedded inside the robot's body 506 that detects the angular velocity of the robot relative to itself and or rotation along the x, y and z axis as well as gyroscope sensor. Gyroscopes measure angular rate and are usually combined with an accelerometer in a common package to allow advanced algorithms like sensor fusion (for orientation estimation in 3D space). In that sense we call them iNEMO (Inertial Modules) or more generally IMU (Inertial Measurement Unit), which can also contain a magnetometer. Typically, IMU is a multi-chip module (MCM) consisting of the 3-Axis gyroscope, the 3-Axis accelerometer and in some event the 3-Axis magnetometer. Such 6-axis or 9-axis motion tracking device combines a 3-axis gyroscope, 3-axis accelerometer, 3-axis magnetometer, and a typically aa digital motion processor. The inertia measurement unit (IMU) takes the input feeds regarding angular velocity data, rotational data and variation in magnetic field data to the central processing component which will determine a variety of the robot's movement. If the robot is upside down, it will remind the child to straighten the robot. If the child is running and holding the robot, the sensor will pick up the traveling speed and orientation of the robot in relation to the child.

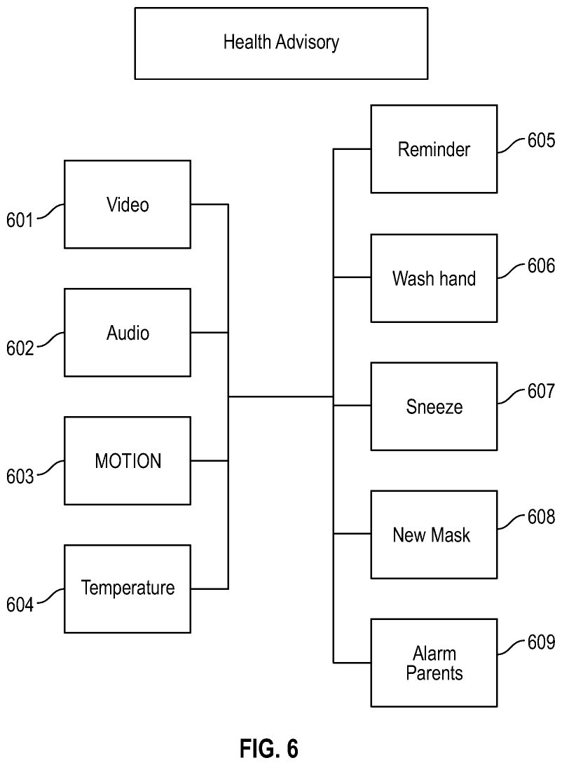

FIG. 6 illustrates an exemplary flow diagram in which the robot platform acts as a self-care and well-being maintenance tool for the child. The platform has the video sensor, audio sensor, IMU sensor, and infrared temperature sensor as a collection of various attributes provided by the children. Specifically, the platform has a camera for which the video analysis module 601 can perform the image analysis. The platform also has an audio analysis module 602 for which sound content can be analyzed. The platform also has a motion sensor module 603 for which the angular, horizontal, and vertical speed of the platform can be detected and analyzed.

The platform also has a temperature sensing module 604 for which the temperature information can be collected. Typical example of a temperature sensing module is an infrared thermometer which is a thermometer which infers temperature from a portion of the thermal radiation sometimes called black-body radiation emitted by the object being measured. They are sometimes called laser thermometers as a laser is used to help aim the thermometer, or non-contact thermometers or temperature guns, to describe the device's ability to measure temperature from a distance. By knowing the amount of infrared energy emitted by the object and its emissivity, the object's temperature can often be determined within a certain range of its actual temperature. Infrared thermometers are a subset of devices known as “thermal radiation thermometers”.

Typical example of audio analysis module 602 is an interdisciplinary subfield of computer science and computational linguistics that develops methodologies and technologies that enable the recognition and translation of spoken language into text by computers.

In one embodiment, the present speech recognition systems require “training” where an individual speaker reads text or isolated vocabulary into the system. The system analyzes the person's specific voice and uses it to fine-tune the recognition of that person's speech, resulting in increased accuracy. In another embodiment, the present invention module does not use training and is called “speaker independent” systems. Systems that use training are called “speaker dependent”.

In one embodiment, the present invention utilizes speech recognition applications including voice user interfaces such as search key words, simple data entry, determining speaker characteristics, speech-to-text processing. In one embodiment, the voice recognition or speaker identification is capable of identifying the speaker, rather than just figuring out what they are saying. Recognizing the speaker can augment the function and realism of the present AI invention which have been trained on a specific person's voice and can be used to authenticate or verify the identity of a speaker as part of the interaction process in children development.

In yet another embodiment, the present voice module considers the fundamental frequency in the voiced sounds of speech which is defined as pitch in part of its voice analysis. The module considers the frequency of the mechanical movement in the glottis as it relates to its physical characteristics. One method employed by the present invention for determining the pitch in a discrete speech signal is to utilize the function of autocorrelation which uses a sample of greater amplitude in an interval defined between two frequencies with amplitude greater than 30% of the initial energy represents the frequency of pitch, and defining the value of frequency as it is related with the sampling frequency.

Furthermore, in one embodiment, the present voice analysis system also provides validated access to the system by an automatic speech recognition subsystem using selection of speech models based on voice input characteristics. In this embodiment, the two-way microphones 152 obtain speech data from the child. The voice data is converted by an A/D component to a digital format. Note that the digital data includes environmental data, which assists the voice analysis module to further discern the speaker's tone, gender, age group, or emotion. The features could be any combination of Mel Frequency Cepstral Coefficients, Filterbank Energies, Log Filterbank Energies, Spectral Subband Centroids, Zero Crossing Rate, Energy Entropy of Energy, Spectral Centroid Spectral Spread, Spectral Entropy, Spectral Flux, Spectral Rolloff, Chroma Vector Chroma Deviation and more. These pluralities of sound features of the speech are used by a classifier module to classify the speech. This classification process is one of several steps of the present invention's speech recognition process. The classifier module uses a database of preference voice profiles of the speaker to match and verify the speaker's voice, and, thus, the speaker's identity. The present voice recognition system employs deep learning models, such as HMM (hidden markov model), GMM (gaussian mixture), GMM-HMM, DNN (deep neural network)-HMM, RNN or a ensemble or hybrid of these models where it is appropriate to achieve the best speech recognition subsystem. These models can decode speeches with their variation based on training, such as initial speaker voice samples, or speaker's voice samples collected over time. Furthermore, the present invention also contemplates, in another embodiment, the voice recognition subsystem will choose the appropriate models based on the speaker's voice sample dataset collected over time. The current invention's AI system utilizes CPU, GPU and the models data stored on the memory to decode the speech, and, similarly, video images.

Collecting on these information, these information harvested can be used to provide self-care and well-being reminders 605 to the children, such as reminding the children to wash hands 606, or when the platform can recognize the children when sneezed 607 and need to wear a mask 608 or if needed the platform will issue an alarm to the parents 609.

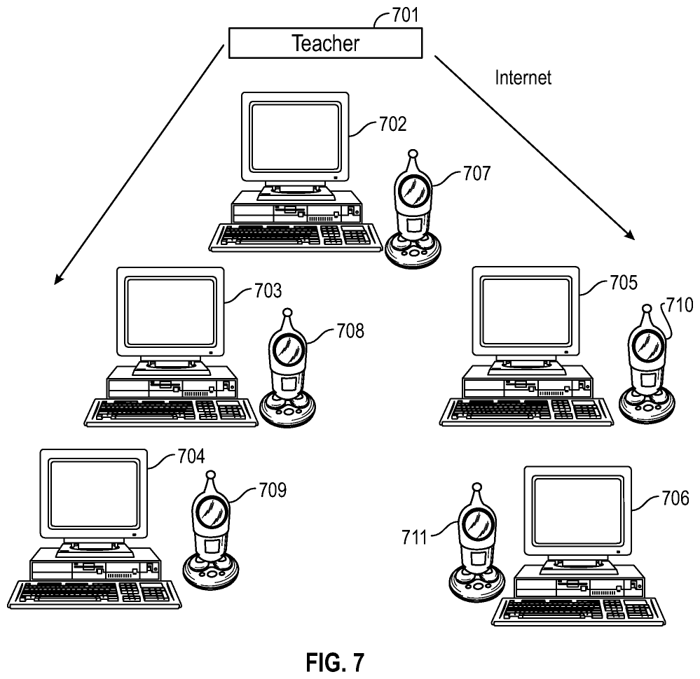

FIG. 7 illustrates an exemplary flow diagram of a network system providing content to registered individual robots. This is the platform's ability to act as an additional aid tool in the context of distant learning. A teacher 701 initiates distant learning with real life stream provides educational content on demand from individual registered robots. Here a teacher actively teaches remote children via a teleconferencing system and using the robots to implement the lessons, or tests or exercises. The children, as he receives the tests or lessons, provides input both the charging pad input reception and as well as provides feedback through the attributes harvested by the different sensors on the platform.

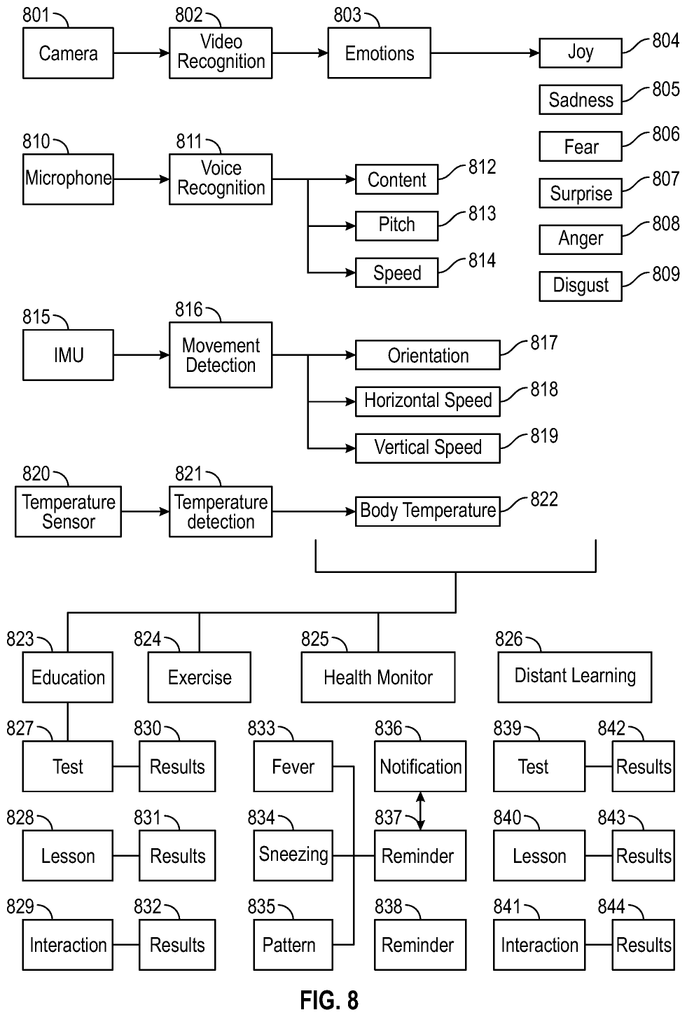

FIG. 8 is a schematic diagram of one aspect of the invention of the function flow of the robotic apparatus. The robot apparatus has at least one camera sensor 801, a microphone (audio sensor) 810, an internal movement sensor (Inertial measurement unit (IMU) sensor) 815 and an infra-red sensor 820. In one embodiment, the camera sensor 801 captures facial expressions of the child and or children via the video recognition module 802 powered by the CPU within the robotic apparatus and determining the emotions 803 of the children. These are the various attributes that video recognition module 802 is able to harvest. The video recognition module 802 is able to determine the emotions 803 as joy 804, sadness 805, fear 806, surprise 807, anger 808 and disgust 809. Humans are used to taking in non-verbal cues from facial emotions. Now computers are also getting better at reading emotions. The emotions can be classified into 7 classes: joy, sadness, fear, surprise, anger, disgust and neutral. Model—which the present invention uses image augmentations to improve model performance. As it is known intuitively, emotion recognition is the process of identifying human emotion which is a skill we as humans take for granted. People vary widely in their accuracy at recognizing the emotions of others. Use of technology to help machines with emotion recognition is a relatively advanced area. Generally, the technology works best if it uses multiple modalities in context. In one embodiment, the preset invention centers its function on automating the recognition of facial expressions from video and spoken expressions from audio.

The accuracy of emotion recognition of children is usually improved when it combines the analysis of children expressions from multimodal forms such as audio and video. The present invention detects different emotion types through the integration of information from facial expressions of the children, body movement and gestures of the children, and speech of the children. In one embodiment, the present invention utilizes the knowledge-based techniques. Specifically, Knowledge-based techniques utilizes domain knowledge and the semantic and syntactic characteristics of children's spoken language in order to detect certain emotion types. One of the advantages of this approach is the accessibility and economy brought about by the large availability of such knowledge-based resources currently available. A limitation of this technique on the other hand, is its inability to handle concept nuances and complex linguistic rules. Knowledge-based techniques can be mainly classified into two categories: dictionary-based and corpus-based approaches. Dictionary-based approaches find opinion or emotion seed words in a dictionary and search for their synonyms and antonyms to expand the initial list of opinions or emotions in children. Corpus-based approaches on the other hand, start with a seed list of opinion or emotion words, and expand the database by finding other words with context-specific characteristics in a large corpus. While corpus-based approaches take into account context, their performance still varies in different domains since a word in one domain can have a different orientation in another domain.

In another embodiment, the present invention utilizes statistical methods. Statistical methods commonly involve the use of supervised machine learning algorithms in which a large set of annotated data is fed into the algorithms for the system to learn and predict the appropriate emotion types. Machine learning algorithms will provide more reasonable classification accuracy. Deep learning, which is under the unsupervised family of machine learning, is also a method to which the emotion analysis module employed in the present invention. The present invention utilizes deep learning algorithms include different architectures of Artificial Neural Network (ANN) such as Convolutional Neural Network (CNN), Long Short-term Memory (LSTM), and Extreme Learning Machine (ELM) to empower the analysis. In yet another embodiment, the present invention utilizes the hybrid approach in emotion detection. Hybrid approaches in emotion recognition utilizes a combination of knowledge-based techniques and statistical methods, which exploit complementary characteristics from both techniques. Because hybrid techniques gain from the benefits offered by both knowledge-based and statistical approaches, it has better classification performance as opposed to employing knowledge-based or statistical methods independently and is the preferred method for the present invention.

In one embodiment, the audio sensor 810 is able to detect the children's speech via the apparatus' voice recognition module 811. Based on the variation in the voice recognition module 811 function, the module is able to discern the voice content of the children 812, or the pitch of the voice of the children 813 and in one embodiment, detect the speed of the speech of the children 814. These are the various attributes that the voice recognition module 811 is able to harvest. In one embodiment, the apparatus has a motion sensor in the form of inertia measurement unit (IMU) sensor 8, the sensor is able to determine the angular moving speed and lateral moving speed and the motion detection module 816 can detect orientation 817 of the robotic apparatus, the module can detect horizontal speed 818 of the robotic apparatus and the module can detect vertical speed 819 of the robotic apparatus. These are the various attributes the motion detection module 816 can harvest. In one embodiment, the apparatus also has an infrared sensor that is powered by a temperature measurement module 821 and such module is able to detect at least the body temperature 822 of the children.

The various emotion states as attributes, and the various voice attributes, and various motion in the movement attributes and attributes are all part of the data set that the robotic apparatus can collect and utilize specifically in one aspect of the invention, the robotic apparatus provides a platform as educational platform 823 to teach and interact with the children. In one embodiment, the platform detects the content of the speech and use it as a keyword for both voice command in launching certain lessors and also as a tag to launch lessons relating to the subject matter identified by the keyword. For example, when the child says “animal”, then platform plays lessons in the animal category. A such, the platform in children education of the present invention intends to replace the outdated system of one-size-fits-all approach and instead using AI technology to harvest attributes so as to garnish the child's attention and effectuate the learning proficiency base on the child's pace and interest.

In one embodiment, the platform emotion detection technology to analyze the performance of each child during the lessons. When the lessons play, the platform uses emotion detection to see if the child is joy with the lesson. In such way, it can analyze if the lesson performs well as compatibility to the child's interest and pace. The platform can also uses this information to rearrange the lessons/content based on how well each child receive the specific lesson base on the emotion detection.

In one embodiment, the platform 823 can deploy tests 827 for the children and analyze the results 830 of the test base on the inputs and base on the attributes harvested. For example, the platform can provide lessons 828 in math or language, and the platform analyzes lesson results 831. The lesson results can also be analyzed also with the attributes harvested. In some other embodiment, the platform can initiate random or calculated interaction 829 to a children and the platform can then analyze the interaction results 832. In one embodiment, the interaction results can be analyzed by the attributes harvested. In one embodiment, the platform 823 can detect the repetition of key words from the children to determine the preferred subject matter of the user and push lessons to the user having topics relating to the preferred subject matter to entice the user to stay interested and engaged. In the same context, the platform 823 will analyze the interaction results 832 and push lessons to the user having topics relating to the preferred subject matter to entice the user to stay interested and engaged. In one embodiment, the platform 823 can deploy tests 827 for the children and analyze the results 830 of the test base on the inputs and base on the attributes harvested. For example, the platform can provide lessons 828 in math or language, and the platform analyzes lesson results 831. The lesson results can also be analyzed also with the attributes harvested. In some other embodiment, the platform can initiate random or calculated interaction 829 to a children and the platform can then analyze the interaction results 832. In one embodiment, the interaction results can be analyzed by the attributes harvested.

In another aspect of the invention, the platform can be used as a self-care and well-being monitor 825 to the children. In one embodiment, the platform can implement a fever check 833 by using the attributes harvested, specifically from the body temperate 822 and in combination of using the data from the attributes such as the emotions 803 of the children if the children is agitated 809 or anger 808. Once determined of the result, the platform can send notification to the parents 836 via email or text messages. In another example, the platform can use facial recognition technology 802 if the children is sneezing 834 and if determined, the platform can send notification 836 and or perform scheduled event such as issue voice reminder 838 to the children cover the face when sneezing or to obtain a tissue paper to wipe the nose. In one embodiment, the platform can perform regular reminders to ask the child to wash hands, depending on the macro circumstances such as during a pandemic. In yet another embodiment, a pre scheduled pattern 835 can be programmed for the platform to detect the need to issue notification 836 or to exercise remedy 837.

In yet another aspect of the invention, the platform provides an excellent tool as an aid to the distant learning field. Specifically, the platform acts as an excellent complimentary tool when teachers implement distant learning through the use of apps, laptops and desktops. Specifically, the standard laptop or ipad does not have the array of sensors to harvest the attributes of the meeting attendees and also lacks the specific input functions provided by the platform. In this context, a wider variety of distant learning tests 839, distant learning lessons 840 can be implemented through the platform and the results can be analyzed using the additional attributes received to aid the teacher. Further, the efficacy of the teaching sessions can be measured using the data received to measure student's response both while the session is ongoing as well as when the data set can be evaluated after the session ended. In yet another aspect of the invention, the platform provides as an excellent tool to implement physical exercise and physical games for the children The attributes collected from the sensors such as the gyroscopic sensor provides an excellent tool for the child to perform physical exercises by holding it and using such tool to train children to do physical exercises. In another example of the embodiment, virtual classrooms can be created where teachers and students are linked on a platform which also links the AI robot of the present invention together. By using the robot's app and education tool platform, teachers can implement their curriculum, utilize the robot's AI assessment tools, and even personalize each robot specifically for each child. This removes the traditional way of teaching the same things to all children without having the ability to customizing lessons to each child's needs.

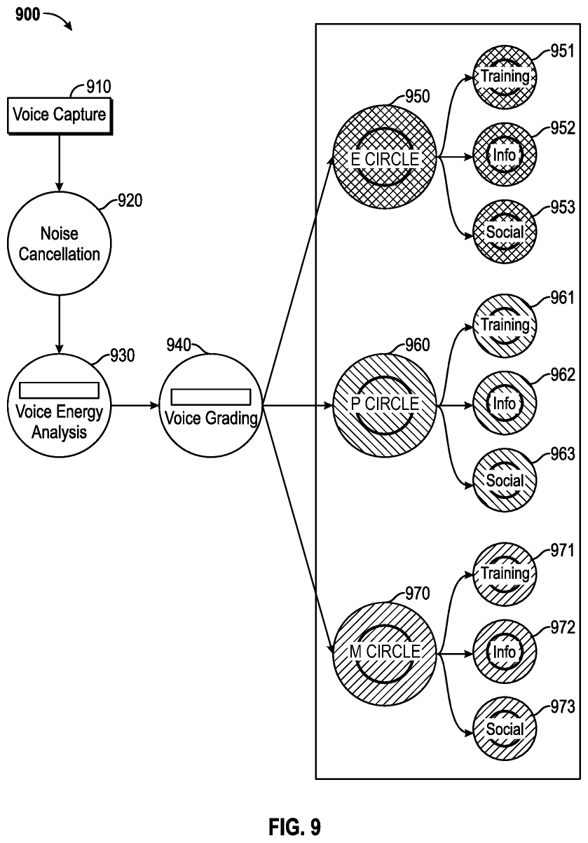

FIG. 9 illustrates an exemplary flowchart of an embodiment 900 of the present invention's unique voice recognition subsystem. In this embodiment, a child's voice is detected and captured by the voice capture component 910 that comprises, among other components, a microphone and a A/D converter. The captured voice is converted into digital format for subsequent analyses. The digital voice data is passed to component 920 to be parsed. This component extracts voice features of the voice data such as environment noises that can be analyzed and give the child's voice the context such as if the child was playing, was at home, was in his study, etc. The environment noises can be removed and only the child's speech is retained where the present invention's feature is only interested in analyzing the content of the child's speech to learn its meaning. In other features, the environment noises are retained and added to a data vector that is passed along to the voice energy analysis component 930. This component analyzes the child's voice frequency and amplitude within the environment noise context by running the data vector through a deep learning neural network, which, depending on the customization level of the embodiment, can be HMM (hidden markov model), GMM (gaussian mixture), GMM-HMM, DNN (deep neural network)-HMM, RNN or an ensemble or hybrid of these models. The analysis outcome is passed to the voice grading or classification component 940, which employs another deep learning neural network and, in this embodiment, grades the personality of the child speaker. In this embodiment, there are three resulting personality types: E, i.e., emotional; P, i.e., planning or organizing; and M, i.e., creative mind. E personality 950 is commonly exhibited by children who often make decisions based on emotions, rather than logical thinking. P personality 960 is commonly exhibited by children who are often very organized and structured, and good at planning activities. M personality 970 is commonly exhibited by children who are creative and often have different approaches to an issue. Over time and interaction with the child, the grading component 940 and the present system can identify the child's personality. Based on the identified personality type, the present system tailors the training or educational content or general information to the identified personality so that the child learns the materials in an optimal way for E Personality Type 951, Personality Type 961 and M Personality Type 971. Furthermore, based on the identified personality, the present system recommends a social group of like-minded children or social groups that complement the child's personality to best help the child's development. In this embodiment, the social group are set according to E Personality Type 952, Personality Type 962 and M Personality Type 972. It is contemplated that in other embodiments there are more personality traits that are fine tuned to provide more specific educational content. In this embodiment, the content group are set according to E Personality Type 953, Personality Type 963 and M Personality Type 973. It is also contemplated that the deep learning personality classification component learns and defines a new category of personalities based on the voice and interaction pattern it learns over time. It, then, provides fine-tuned educational content based on such intelligence.

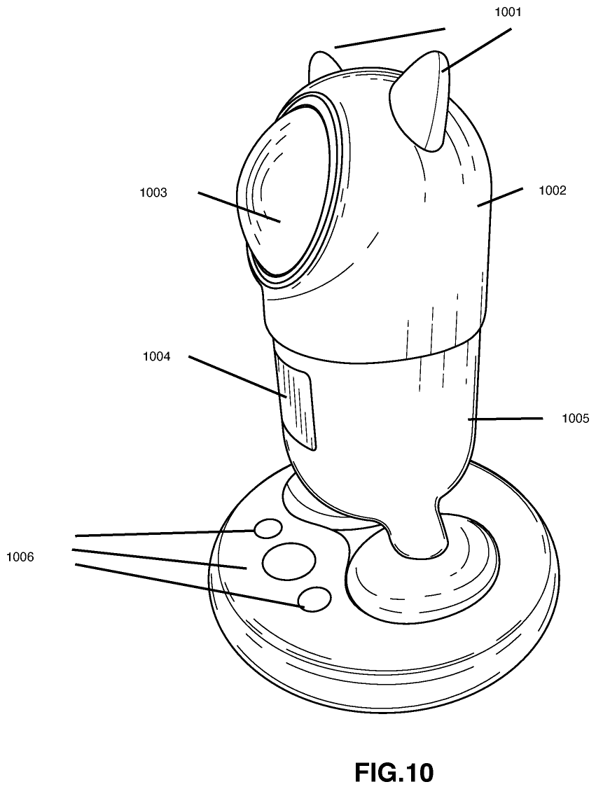

FIG. 10 illustrates an embodiment of the current invention of the children education apparatus of the present invention. The capsule shape body has the upper portion 1002 and the lower portion 1005. The upper portion has the dome like part 1003 that extends out of the upper body 1003 which houses the digital camera. The upper body is covered by flexible part 1001 which in this embodiment is comprised of 2 ear like part that looks like a pair of standing triangle or pyramid looking part for decorative purpose to look like ears. The lower part 1005 which has the digital display 1004. Also presented are the input buttons 1006 on the charging pad.

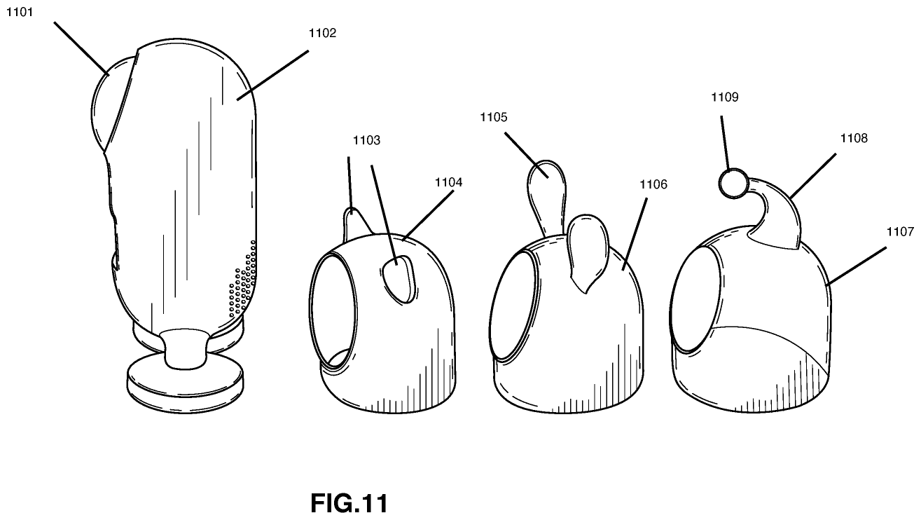

FIG. 11 illustrates an embodiment of the current invention of the children education apparatus of the present invention. The capsule shape body 1102 has a dome like part 1101 that extends out of the capsule shape body 1102 which houses the digital camera. The upper body of the capsule shape body 1102 is covered by flexible part 1004, or 1105 or 1106. Flexible part 1104 has a pair of ears looking part that are formed as revere standing triangle 1104. In another embodiment, flexible part 1108 has a pair of ears looking part that are formed as tear drop part 1105. In another embodiment, flexible part 1107 has a standing triangle part 1108 that has a circular part 1109 attached at the tip of the triangle part 1108.

In one aspect of the invention, an apparatus for educating children is disclosed comprising: an anime-like body comprises an upper and lower body and two feet upon which the anime-like body stands; wherein the upper body is outfitted with an interchangeable cap that comprises two decorative ears; wherein the upper body further comprises a prominent camera that is connected and controlled by a central processing system disposed inside the anime-like body; wherein the camera further comprises a facial recognition system that triggers the camera to capture a motion video; wherein the central processing system processes the motion video; wherein the lower body further comprises a LCD for displaying educational content, warning or feedback messages, that is controlled by the central processing system; wherein the apparatus further comprises a charging pad upon that the anime-like body stands while it is being charged; wherein the charging pad further comprises a home button, a right input button, and left input button;

wherein the home, left and right button is made of clear and soft material that covers an LED light array underneath; wherein the LED light array is controlled by the central processing system to display different colors as visual cues; wherein the charging pad further comprises a shaped depression that one-way fits the two feet; wherein the shaped depression further comprises two sets of electrical pins disposed where the two feet to be set; wherein the two feet further comprises charging plates at the two feet's bottoms; wherein the two sets of electrical pins engage the charging plates; wherein the two feet further comprises two two-way audio unit to pick up and deliver sound content; wherein the LCD displays a question as the apparatus requests a respond through the two-way audio unit; wherein pressing one of the home, left, or right button answers the question; wherein the apparatus further comprises a first two-way wireless communication counterpart; wherein the charging pad further comprises a second two-way wireless communication counterpart; wherein the charging pad further comprises a power socket that receives a DC jack; wherein the apparatus further comprises an inertial measurement unit (IMU) disposed inside the apparatus.

In one embodiment, the sounds received by the two-way audio unit are processed by an integrated circuit (IC) that is a system on chip (SOC) ICB. In one embodiment, the interchangeable cap is a protective cover to protect the upper body. In one embodiment, the interchangeable cap has an appealing design, shape, and color to attract and retain young children's attention. In one embodiment, the apparatus further comprises an electromagnetic inductive charging component. In one embodiment, the communication signals forth and back between the apparatus and the charging pad are transmitted via a wireless component. In one embodiment, the charging pad further comprises at least one magnet that holds the two feet in place and keeps the apparatus standing while it's being charged. In one embodiment, the IMU is comprised of a gyroscope, an accelerometer and a magnetometer.

In another aspect of the invention, a children education apparatus is disclosed comprising: a capsule shape housing unit having a detachable cover part and a feet part wherein the capsule shape housing unit is comprised of an upper capsule part and a lower capsule part wherein the upper capsule part having a forward facing surface and a rearward facing surface wherein the upper capsule part is further comprised of a hemispherical dome part wherein the hemispherical dome part is positioned in vertical fashion along the forward facing surface; the hemispherical dome part houses a digital camera and the detachable cover part is comprised of flexible material and wraps around the upper capsule part in its entirety except wherein the detachable cover part has an opening allowing the hemispherical dome part to protrude through the detachable cover part and the detachable cover part further comprising a vertical standing part for decorative purpose; the lower capsule part is connected of the feet part wherein the feet part is comprised of a first foot and a second foot and the first foot and second foot are of equal length, wherein the lower capsule part further comprises a digital display wherein the digital display is rectangular in shape and wherein the digital display is a LCD, wherein the lower capsule part further comprises at least one microphone, at least a speaker, wherein the capsule shape housing unit further comprises a inertia momentum unit, at least one battery, a computing processing unit. In one embodiment, the children education apparatus unit further comprise a base apparatus wherein the base apparatus comprises a battery charging unit and a recess to receive the first foot and the second feet and charges the battery when the first foot and the second foot rests on the recess; the base apparatus further comprising a plurality of input buttons wherein the input buttons provides input controls the children education apparatus. In one embodiment, the input buttons provides input controls to the children education apparatus when the first foot and the second foot rests on the base apparatus. In one embodiment, the input buttons provides input controls to the children education apparatus when the first foot and the second foot are not resting on the base apparatus. In one embodiment, the vertical standing part is comprised of 2 triangular shape standing parts. In one embodiment, the vertical part is comprised of 2 vertical standing reverse teardrop shape parts. In one embodiment, the vertical part is comprised of a triangular shape standing part having a circular ball shape part attached to the at the tip of the triangular shape standing part. In one embodiment, the capsule housing unit further comprises a temperature sensor.

In yet another aspect of the invention, a children education apparatus is disclosed comprising a central processing unit, an inertia momentum unit, a battery, a camera, a LCD unit, and an education platform power by the central processing unit wherein the education platform is comprised of plurality of children education content and delivers the plurality of children education content to the LCD unit where the education platform detects a movement of the children education apparatus via the inertia momentum unit and determines which one of the plurality of children education content to the LCD.

In another aspect of the invention, a children education apparatus is disclosed comprising a central processing unit, an inertia momentum unit, a battery, a camera, a microphone, a temperature sensor, a LCD unit, and an education platform power by the central processing unit wherein the education platform is comprised of plurality of children education content and delivers the plurality of children education content to the LCD unit where the education platform is comprised of a video data analysis module, a voice data analysis module, a movement data analysis module and a temperature data analysis module wherein the camera records one or more video data and the microphone records one or more records one or more audio data and the inertia momentum unit records one or more movement data and the temperature sensor records one or more temperature data. In one embodiment, the video data analysis module analyzes the video data and determines which one of the plurality of children education content to deliver to the LCD. In one embodiment, the audio data analysis module analyzes the audio data and determines which one of the plurality of children education content to deliver to the LCD. In one embodiment, the temperature data analysis module analyzes the temperature data and determines which one of the plurality of children education content to deliver to the LCD. In one embodiment, the movement data analysis module analyzes the movement data and determines which one of the plurality of children education content to deliver to the LCD. In one embodiment, the video data analysis module analyzes the video data and determines an emotion characterization of a user and determines which one of the plurality of children education content to deliver to the LCD base on the emotion characterization. In one embodiment, the audio data analysis module analyzes the audio data and determines an emotion characterization of a user and determines which one of the plurality of children education content to deliver to the LCD base on the emotion characterization.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to illustrate aspects of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. 112(f) unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from.

The embodiments of the invention in which an exclusive property or privilege is claimed are defined as follows:

1. An apparatus for educating children comprising:
An anime-like body comprises an upper and lower body and two feet upon which said anime-like body stands;
wherein said upper body is outfitted with an interchangeable cap that comprises two decorative ears;
wherein said upper body further comprises a prominent camera that is connected and controlled by a central processing system disposed inside said anime-like body;
wherein said camera further comprises a facial recognition system that triggers said camera to capture a motion video;
wherein said central processing system processes said motion video;
wherein said lower body further comprises a LCD for displaying educational content, warning or feedback messages, that is controlled by said central processing system;
wherein said apparatus further comprises a charging pad upon that said anime-like body stands while it is being charged;
wherein said charging pad further comprises a home button, a right input button, and left input button;
wherein said home, left and right button is made of clear and soft material that covers an LED light array underneath;
wherein said LED light array is controlled by said central processing system to display different colors as visual cues;
wherein said charging pad further comprises a shaped depression that one-way fits said two feet;
wherein said shaped depression further comprises two sets of electrical pins disposed where said two feet to be set;
wherein said two feet further comprises charging plates at said two feet's bottoms;
wherein said two sets of electrical pins engage said charging plates;
wherein said two feet further comprises two two-way audio unit to pick up and deliver sound content;
wherein said LCD displays a question as said apparatus requests a respond through said two-way microphones;
wherein pressing one of said home, left, or right button answers said question;
wherein said apparatus further comprises a first two-way wireless communication counterpart;
wherein said charging pad further comprises a second two-way wireless communication counterpart;
wherein said charging pad further comprises a power socket that receives a DC jack;
wherein said apparatus further comprises an inertial measurement unit (IMU) disposed inside said apparatus;

2. The apparatus of claim 1, wherein sounds received by said two-way audio unit are processed by an integrated circuit (IC) that is a system on chip (SOC) ICB.

3. The apparatus of claim 1, wherein said interchangeable cap is a protective cover to protect said upper body.

4. The apparatus of claim 1, wherein said interchangeable cap has an appealing design, shape, and color to attract and retain young children's attention.

5. The apparatus of claim 1 further comprises an electromagnetic inductive charging component.

6. The apparatus of claim 5, wherein communication signals forth and back between said apparatus and said charging pad are transmitted via a wireless component.

7. The apparatus of claim 1, wherein said charging pad further comprises at least one magnet that holds said two feet in place and keeps said apparatus standing while it's being charged.

8. The apparatus of claim 1, wherein said IMU comprises a gyroscope, an accelerometer and a magnetometer.

9. An children education apparatus comprising:
A capsule shape housing unit having a detachable cover part and a feet part wherein said capsule shape housing unit is comprised of an upper capsule part and a lower capsule part wherein said upper capsule part having a forward facing surface and a rearward facing surface wherein said upper capsule part is further comprised of a hemispherical dome part wherein said hemispherical dome part is positioned in vertical fashion along said forward facing surface; said hemispherical dome part houses a digital camera and said detachable cover part is comprised of flexible material and wraps around said upper capsule part in its entirety except wherein said detachable cover part has an opening allowing said hemispherical dome part to protrude through said detachable cover part and said detachable cover part further comprising a vertical standing part for decorative purpose; said lower capsule part is connected of said feet part wherein said feet part is comprised of a first foot and a second foot and said first foot and second foot are of equal length, wherein said lower capsule part further comprises a digital display wherein said digital display is rectangular in shape and wherein said digital display is a LCD, wherein said lower capsule part further comprises at least one microphone, at least a speaker, wherein said capsule shape housing unit further comprises a inertia momentum unit, at least one battery, a computing processing unit.

10. The apparatus of claim 9, wherein said children education apparatus unit further comprise a base apparatus wherein said base apparatus comprises a battery charging unit and a recess to received said first foot and said second feet and charges said battery when said first foot and said second foot rests on said recess; said base apparatus further comprising a plurality of input buttons wherein said input buttons provides input controls said children education apparatus.

11. The apparatus of claim 10, wherein said input buttons provides input controls to said children education apparatus when said first foot and said second foot rests on said base apparatus.

12. The apparatus of claim 11, wherein said input buttons provides input controls to said children education apparatus when said first foot and said second foot are not resting on said base apparatus.

13. The apparatus of claim 12, wherein said vertical standing part is comprised of 2 triangular shape standing parts.

14. The apparatus of claim 13, wherein said vertical part is comprised of 2 vertical standing reverse teardrop shape parts.

15. The apparatus of claim 14, wherein said vertical part is comprised of a triangular shape standing part having a circular ball shape part attached to said at the tip of said triangular shape standing part.

16. The apparatus of claim 9, wherein said capsule housing unit further comprises a temperature sensor.

17. A children education apparatus comprising a central processing unit, an inertia momentum unit, a battery, a camera, a LCD unit, and an education platform power by said central processing unit wherein said education platform is comprised of plurality of children education content and delivers said plurality of children education content to said LCD unit where said education platform detects a movement of said children education apparatus via said inertia momentum unit and determines which one of said plurality of children education content to said LCD.

18. A children education apparatus comprising a central processing unit, an inertia momentum unit, a battery, a camera, a microphone, a temperature sensor, a LCD unit, and an education platform power by said central processing unit wherein said education platform is comprised of plurality of children education content and delivers said plurality of children education content to said LCD unit where said education platform is comprised of a video data analysis module, a voice data analysis module, a movement data analysis module and a temperature data analysis module wherein said camera records one or more video data and said microphone records one or more records one or more audio data and said inertia momentum unit records one or more movement data and said temperature sensor records one or more temperature data.

19. The claim of 18 wherein and said video data analysis module analyzes said video data and determines which one of said plurality of children education content to deliver to said LCD.

20. The claim of 18 wherein and said audio data analysis module analyzes said audio data and determines which one of said plurality of children education content to deliver to said LCD.

21. The claim of 18 wherein and said temperature data analysis module analyzes said temperature data and determines which one of said plurality of children education content to deliver to said LCD.

22. The claim of 18 wherein and said movement data analysis module analyzes said movement data and determines which one of said plurality of children education content to deliver to said LCD.

23. The claim of 19 wherein and said video data analysis module analyzes said video data and determines an emotion characterization of a user and determines which one of said plurality of children education content to deliver to said LCD base on said emotion characterization.

24. The claim of 20 wherein and said audio data analysis module analyzes said audio data and determines an emotion characterization of a user and determines which one of said plurality of children education content to deliver to said LCD base on said emotion characterization.
